(19) United States
(12) Patent Application Publication
(10) Pub. No.: US 2007/0248564 A1
(43) Pub. Date: Oct. 25, 2007

(54) FORMULATION OF SODIUM POLYSTYRENE SULFONATE SUSPENSION FOR THE TREATMENT OF HYPERKALEMIA

(75) Inventors: Randall S. Wilson, Dublin, OH (US); Andrea M. McPhillips, Dublin, OH (US)

Correspondence Address: PEARNE & GORDON LLP, 1801 EAST 9TH STREET, SUITE 1200, CLEVELAND, OH, 44114-3108, US

(73) Assignee: ROXANE LABORATORIES, INC., Columbus, OH (US)

(21) Appl. No.: 11/740,068

(22) Filed: Apr. 25, 2007

Related U.S. Application Data
(60) Provisional application No. 60/794,895, filed on Apr. 25, 2006.

(51) Int. Cl. A61K 31/74

(52) U.S. Cl. 424/78.1

(57) ABSTRACT

This invention relates to a stable, sorbitol free suspension formulation of sodium polystyrene sulfonate.

### CROSS-REFERENCE TO RELATED APPLICATIONS

This application claims the benefit of U.S. Provisional Patent Application No. 60/794,895 filed Apr. 25, 2006, which is incorporated herein by reference in its entirety.

### FIELD OF THE INVENTION

This invention relates to a stable, sorbitol free suspension formulation of sodium polystyrene sulfonate.

### BACKGROUND OF THE INVENTION

Potassium has many functions in the body. It helps regulate the activity of all muscle tissue-smooth muscles (such as the muscles in the intestines), the muscles of the heart, and skeletal muscles. Potassium also plays a part in the enzymatic reactions involved in digestion and other metabolisms of the body. Potassium further plays a role in homeostasis, the mechanism used by the body to maintain a balance between the many electrical and chemical processes of the body.

Almost all (98%) of the potassium in the body is found inside cells (intracellular). Only about 2% occurs in the fluids outside of the cells (extracellular). Potassium can move into and out of cells as necessary to maintain the proper balance in the body.

Blood tests reveal the extracellular potassium levels and are not indicative of the amount of intracellular potassium. Movement of potassium into or out of the cells can change the blood potassium level (serum potassium) when there is no change in the overall amount of potassium in the body.

Hyperkalemia occurs when the level of potassium in the bloodstream is higher than normal. This may be related to an increase in total body potassium or excessive release of potassium from the cells of the body into the bloodstream.

Sodium polystyrene sulfonate suspension, USP is an approved pharmaceutical product for the treatment of hyperkalemia. It can be administered orally or rectally (by enema). One current marketed formulation consists of several ingredients, including sorbitol solution, USP. This ingredient is used as a vehicle in the formulation. High density liquids such as sorbitol are often used in suspension formulations to increase physical stability. Recent literature indicates potential harmful gastrointestinal side effects with the concomitant use of sorbitol and sodium polystyrene sulfonate such as bleeding, hematochezia, colonic perforation, colonic necrosis and/or serpiginous ulcers. (Chaudhury, et al., American Journal of Kidney Diseases, Vol. 30, No. 1 (July) 1997: pp. 120-122; Gerstman, et al., American Journal of Kidney Diseases, Vol. XX, No. 2 (August) 1992: pp. 159-161; Gardiner, Can J. Gastroenterol, Vol. 11, No. 7 (October) 1997: pp. 573-577.)

Accordingly, development of a sorbitol-free sodium polystyrene sulfonate suspension which is physically and chemically stable for the treatment of hyperkalemia is desirable.

### SUMMARY OF THE INVENTION

In accordance with the present invention, a stable and manufacturable pharmaceutically elegant sorbitol-free suspension of sodium polystyrene sulfonate is provided.

According to one aspect of the present invention, a sodium polystyrene sulfonate suspension utilizes water in place of sorbitol to form a suspension which has good chemical and physical stability without the detrimental side effects attributed to sorbitol.

A further aspect of the invention relates to a method of treating hyperkalemia in a patient in need thereof by administration of a sorbitol-free formulation of sodium polystyrene sulfonate.

A further aspect of the invention relates to a pharmaceutically acceptable formulation of sodium polystyrene sulfonate in a suspension form for treatment of hyperkalemia.

A further aspect of the invention relates to a ready-to-use, pharmaceutically acceptable formulation of sodium polystyrene sulfonate in a suspension form for treatment of hyperkalemia.

Another aspect of the invention relates to a pharmaceutically acceptable formulation of sodium polystyrene sulfonate in a suspension form for treatment of hyperkalemia wherein the formulation is for oral administration.

Another aspect of the invention relates to a pharmaceutically acceptable formulation of sodium polystyrene sulfonate in a suspension form for treatment of hyperkalemia wherein the formulation is for rectal administration.

These and other aspect and objects of the invention will become readily apparent upon reading and understanding of the specification and claims of the application.

### DETAILED DESCRIPTION OF THE INVENTION

Sodium polystyrene sulfonate is typically administered to patients suffering from hyperkalemia. Sodium polystyrene sulfonate is a benzene, diethenyl-, polymer with ethenylbenzene, sulfonated, sodium salt and has the following structure:

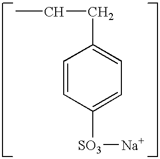

The sodium polystyrene sulfonate exists as a cation exchange resin and is typically administered as an oral solution or in an enema. As the resin passes along the intestine after oral administration or is retained in the colon by rectal administration, the sodium ions are partially released and replaced by potassium ions. For the most part, this action occurs in the large intestine, which excretes potassium ions to a greater extent than does the small intestine.

A pharmaceutically acceptable suspension of sodium polystyrene sulfonate has a sodium concentration of 15.0 g per 60.0 mL. According to the USP, a sodium polystyrene sulfonate suspension is a suspension of sodium polystyrene sulfonate in an aqueous vehicle that may contain suitable suspending or stabilizing agents. The sodium polystyrene sulfonate suspension exchanges not less that 110 mg and not more than 135 mg of potassium for each gram of the labeled amount of sodium polystyrene sulfonate. Other indications may exist or become evident which require differing concentrations or exchange ratios. It will be apparent to one of ordinary skill in the art in view of the present disclosure that the formulation of the invention will be suitable for use at differing concentrations and to provide different levels of exchange activity.

The sorbitol-free sodium polystyrene sulfonate formulation of the present invention contains a number of pharmaceutically acceptable adjuvants including, but not limited to, one or more solvents/vehicles, preservatives, suspending agents, sweetening agents, buffers/pH adjusters, and flavoring agents. In addition, other ingredients such as wetting agents, protective colloids and colorants can be added to the formulation.

The following Table represents one embodiment of the sorbitol free sodium polystyrene sulfonate formulation according to the present invention:

TABLE 1

| Sodium Polystyrene Sulfonate Suspension | Quantitative Composition |
|---|---|
| Sodium Polystyrene Sulfonate | 15.0 g/60.0 mL |
| Solvent/vehicle | 0–99% w/v |
| Preservative | 0–5% |
| Suspending Agent | 0–50% w/v |
| Sweetening Agent | 0–5% w/v |
| Buffers/pH Adjusters | 0–2% w/v |
| Flavors | 0–3% v/v |

A further embodiment of the sorbitol free suspension is represented in Table 2 below:

TABLE 2

| Sodium Polystyrene | Quantitative | | |
|---|---|---|---|
| Sulfonate Suspension | Composition | | Function |
| Sodium Polystyrene Sulfonate | 15.0 g/60.0 mL | | Active Ingredient |
| Propylene Glycol, USP | 0–25% | w/v | Solvent/Vehicle |
| Methylparaben, NF | 0–0.5% | w/v | Preservative |
| Propylparaben, NF | 0–0.5% | w/v | Preservative |
| Veegum (Magnesium | 0–5% | w/v | Suspending Agent |
| Aluminum Silicate, NF) | | | |
| Saccharin Sodium, USP | 0–5% | w/v | Sweetening Agent |
| (Saccharin Soluble) | | | |
| Citric Acid, USP (Anhydrous, | 0–2% | w/v | pH Adjustment agent |
| Fine Gran.) | | | |
| Caramel Concentrate XBF- | 0–3% | v/v | Flavor |
| 700103 | | | |
| Cherry Flavor, Imitation F- | 0–3% | v/v | Flavor |
| 1311 | | | |

| | | |
|---|---|---|
| Water, Purified USP | QS | Vehicle |

Another embodiment of the invention is represented in Table 3, below:

TABLE 3

| Sodium Polystyrene | | | |
|---|---|---|---|
| Sulfonate Suspension, USP | Quantitative | | |
| 15 g/60.0 mL | Composition | | Function |
| Amberlite IRP 69 (Sodium | 15.0 | g | Active Ingredient |
| Polystyrene Sulfonate | | | |
| Suspension, USP) | | | |
| Propylene Glycol, USP | 10% | w/v | Solvent/Vehicle |
| Methylparaben, NF | 0.18% | w/v | Preservative |
| Propylparaben, NF | 0.02% | w/v | Preservative |
| Veegum (Magnesium | 1.75% | w/v | Suspending Agent |
| Aluminum Silicate, NF) | | | |
| Saccharin Sodium, USP | 0.025% | w/v | Sweetening Agent |
| (Saccharin Soluble) | | | |
| Citric Acid, USP (Anhydrous, | 0.125% | w/v | pH Adjustment agent |
| Fine Gran.) | | | |
| Caramel Concentrate XBF- | 0.55% | v/v | Flavor |
| 700103 | | | |
| Cherry Flavor, Imitation F- | 0.2% | v/v | Flavor |
| 1311 | | | |

| | | |
|---|---|---|
| Water, Purified USP | QS | Vehicle |

Components in the previously outlined formulation may be substituted including both grades of the raw drug material as well as additives. Various combinations of pharmaceutical additives may be used in the formulation which provide equivalent performance to the above formulation, or are added to the formulation to perform special functions. Examples of substitute materials are outlined in the following tables:

Raw Drug Materials:

For the sodium polystyrene sulfonate component of the formulation, alternative grades may be used in the formulation as long as they are functionally equivalent to the USP.

Pharmaceutical Solvent/Vehicles:

Various solvent/vehicles (collectively referred to as “vehicles”) can be used to carry the active ingredient and other adjuvants in the formulation including, but not limited to, one or more of purified water, propylene glycol, glycerin, alcohol, polyethylene glycol, polyglyceryl-6 oleate and mixtures thereof.

Pharmaceutical Suspending Agents/Protective Colloids:

A variety of pharmaceutically acceptable suspending agents/protective colloids (collectively referred to as “suspending agent”) can be used according to the present invention as long as they are not detrimental to the functionality of the formulation, including the stability of the formulation. The following is a representative sample of the types of suspending agents which are useable in combination with sodium polystyrene sulfonate to form stable, sorbitol free formulations according to the invention:

Gums: acacia, agar, carrageen, guar, karaya, locust bean, pectin, propylene glycol alginate, sodium alginate, tragacanth, and xanthan gums.

Cellulosics: carboxymethyl cellulose sodium, microcrystalline cellulose/carboxymethyl cellulose, hydroxyethyl cellulose, and hydroxypropyl methyl cellulose.

Clays: colloidal aluminum silicate, magnesium silicate and magnesium aluminum silicate.

Others: Carbomer NF, gelatin, polyethylene glycols, and lecithin.

Preservatives:

Preservatives which may be utilized in the formulation of the invention include, but are not limited to, one or more of:

Parabens, sorbic acid, thimerosal, quaternary ammonium salts, benzyl alcohol, benzoic acid, phenylethanol, and chlorhexidine gluconate.

Additional Additives:

Additional conventional pharmaceutical additives which are not detrimental to the stability or other pharmaceutical activity of the formulation may be added and can include various flavorings, colorants, wetting agents (such as, for example, docusate sodium, sodium lauryl sulfate, polysorbates, polxamers), pH control agents and buffers (such as citrates and phosphates).

Manufacturing for Suspension Systems

Conventional incorporation of powders, particularly suspending agents takes place by the slow addition of the material under heat and long periods of recirculation. Heat and recirculation are required in order to completely hydrate the suspending agent and provide enough force to break up agglomerates. This process may take up to 6 hours to complete. In the manufacturing process of the present application, a high shear powder disperser is used to incorporate a suspending agent (such as magnesium aluminum silicate) and sodium polystyrene sulfonate into the suspension. This high shear powder disperser has a rotor-stator design. The rotor creates a liquid ring creating significant vacuum. This vacuum draws the powder from the hopper of the disperser into the reactor head. The powder is introduced into the liquid under high shear before hydration takes place, allowing for a smooth and consistent dispersion. In addition, recirculation times are greatly reduced. By utilizing the rotor-stator system in preparing the sodium polystyrene sulfonate formulation of the present invention, enhanced hydration and drug distribution are realized for the formulation.

### FORMULATION EXAMPLE

The following example represents the manufacturing process by which the formulation depicted in Table 3 above was made. The example below represents one process for preparing a formulation according to the invention. The manufacturing method in the example is merely representative of a formulating method and it is not intended to exclude obvious variants including variations in the order of addition, mixing times, concentrations of excipients and the like which may be changed in order to optimize the manufacturing process.

Tank Preparation and Charging

A tank is prepared for manufacturing utilizing current procedures. A total of 1000.0 kg water, purified, USP is then added to the tank.

Ingredient Addition

Veegum Dispersion

A High Intensity Disperser using a 1.5 mm rotor and 1.5 mm stator is prepared. Veegum (Magnesium Aluminum Silicate, NF) is slowly dispersed into the water, purified USP with intense mixing and recirculated for a minimum of 30 minutes with intense mixing.

Preservative Side Mix

Propylene glycol, USP (170.0 kg) is added to a tank. Methylparaben, NF and propylparaben, NF are added to the tank with intense mixing until dissolved. Once dissolved, the paraben solution is added to the main manufacturing tank and mixed for a minimum of 10 minutes with standard mixing.

Amberlite IRP 69 Dispersion

Amberlite IRP 69 (Sodium Polystyrene Sulfonate, USP) is dispersed in the main manufacturing tank with intense mixing and recirculation through a pump. Once added, the Amberlite IPR 69 (Sodium Polystyrene Sulfonate Suspension, USP) is dispersed for a minimum of 35 minutes with continued intense mixing and recirculation through a pump. Recirculation is discontinued and the solution is mixed for a minimum of 30 minutes with intense mixing.

Saccharin Side Mix

A stockpot is prepared and 4.5 kg of water is added. Saccharin sodium, USP (Saccharin Soluble) is added to the water and dissolved with standard mixing. Once dissolved, the solution is added to the main manufacturing tank and mixed for a minimum of 10 minutes with standard mixing.

Citric Acid Side Mix

A stockpot is prepared and 4.5 kg of water, purified USP is added. Citric acid, USP (anhydrous, fine gran.) is added to the tank and dissolved with standard mixing. Once dissolved, the solution is added to the main manufacturing tank and mixed for a minimum of 10 minutes with standard mixing.

Flavor Addition

Caramel concentrate, XBF-700103 and cherry flavor, imitation, F1311 are added to the manufacturing tank with standard mixing and mixed for a minimum of 10 minutes.

QS Step

The tank is QS to a weight of 1870.0 kg with water, purified USP and mixed for a minimum of 30 minutes with standard mixing.

Transfer and Packaging

The solution is then transferred with standard mixing to a storage tank through a pump equipped with a #20 mesh cartridge screen and packaged into appropriate containers.

Commercial scale formulations manufactured utilizing Example 3 formulation above were stored under the following conditions:

TABLE 4

| Batch No. | Storage Conditions | Package Configurations |
|---|---|---|
| 1 | 25° C./60% RH | Bottle of 500 mL |
| | Upright and Side | 60 mL Unit Dose |
| | 40° C./75% RH | |
| | Upright and side | |
| 2 | 25° C./60% RH | 120 mL Enema |
| | Upright and side | 200 mL Enema |
| | 40° C./75% RH | |
| | Upright and side | |

RH: Relative Humidity

Stability conditions of 25° C./60% RH and 40° C./75% RH are typical storage conditions for pharmaceutical products according to FDA Stability Guidance.

Samples were tested for physical and chemical stability throughout their storage duration including appearance, resuspendability, sodium content, pH, potassium exchange capacity, paraben assay and microbiological testing for product stored at upright and side.

Results of the physical stability parameters of resuspendability/appearance, which are key to the suspension formulation, are summarized in the following tables:

TABLE 5

| | Resuspendability | Batch No. 1 | | Batch No. 2 | |
|---|---|---|---|---|---|
| Condition | Test | Upright | Side | Upright | Side |
| Initial | | Pass | Pass | Pass | Pass |
| 1 mo 40° C./ | | Pass | Pass | Pass | Pass |
| 75% RH | | | | | |
| 2 mo 40° C./ | | Pass | Pass | Pass | Pass |
| 75% RH | | | | | |
| 3 mo 40° C./ | | Pass | Pass | Pass | Pass |
| 75% RH | | | | | |
| 3 mo 25° C./ | | Pass | Pass | Pass | Pass |
| 60% RH | | | | | |
| 6 mo 25° C./ | | Pass | Pass | Pass | Pass |
| 60% RH | | | | | |

RH: Relative Humidity
Pass: Resuspends on shaking well (visual)

TABLE 6

| | Resuspendability | Batch No. 1 | | Batch No. 2 | |
|---|---|---|---|---|---|
| Condition | Test | Upright | Side | Upright | Side |
| Initial | | MS | MS | MS | MS |
| 1 mo 40° C./ | | NC | NC | NC | NC |
| 75% RH | | | | | |
| 2 mo 40° C./ | | NC | NC | NC | NC |
| 75% RH | | | | | |
| 3 mo 40° C./ | | NC | NC | NC | NC |
| 75% RH | | | | | |
| 3 mo 25° C./ | | NC | NC | NC | NC |
| 60% RH | | | | | |
| 6 mo 25° C./ | | NC | NC | NC | NC |
| 60% RH | | | | | |

RH: Relative Humidity
MS: Meets specification (Amber, smooth suspension; visual)
NC: No Change

As can be seen from the above data, the suspension remained stable throughout storage for 6 months at room temperature and 3 months at accelerated stability conditions.

The test standards used for establishing whether the packaged sodium polystyrene sulfonate products are acceptable are set forth in the following Table 7:

TABLE 7

| | | Release Acceptance | Stability Acceptance |
|---|---|---|---|
| Test | Analytical Procedure | Criteria | Criteria |
| Description | Visual | Amber, smooth | Amber, smooth |
| | | suspension | suspension |
| Identification A | | Imparts a yellow color | N/A |
| | | to non-luminous flame.¹ | |
| Identification B | | IR spectrum exhibits | N/A |
| | | maxima only at the | |
| | | same wavelengths of an | |
| | | approved lot of | |
| | | Amberlite IRP 69 Raw | |
| | | Material.² | |
| pH | USP <791> | 4.0 to 7.5 | 4.0 to 7.5 |
| Density | | 1.10 + 0.03 g/mL | N/A |
| Alcohol | USP <611> Method II | NMT 0.3% | N/A |
| | Acetone (IS) | | |
| Solids Content | | 26.0 + 5.0% | N/A |
| Potassium Exchange | USP | One (1) gram equivalent | One (1) gram equivalent |
| Capacity | | of sodium polystyrene | of sodium polystyrene |
| | | sulfonate exchanges not | sulfonate exchanges not |
| | | less than 110 mg and | less than 110 mg and |
| | | not more that 1.35 mg | not more that 1.35 mg |
| | | of potassium. | of potassium. |
| Sodium Content | USP | 2.35 to 2.88% w/v | 2.35 to 2.88% w/v |
| | | (equivalent to 9.4 to | (equivalent to 9.4 to |
| | | 11.5 w/w in the labeled | 11.5 w/w in the labeled |
| | | content of sodium | content of sodium |
| | | polystyrene sulfonate) | polystyrene sulfonate) |
| Assay Methylparaben | | 80.0 to 120.0% of the | 60.0 to 120.0% of the |
| | | labeled amount of | labeled amount of |
| | | methylparaben, 0.18% | methylbaraben, 0.18% |
| | | w/v. | w/v. |
| Assay Propylparaben | | 80.0 to 120.0% of the | 60.0 to 120.0% of the |
| | | labeled amount of | labeled amount of |
| | | propylparaben, 0.02% | propylparaben, 0.02% |
| | | w/v. | w/v. |

¹Combine a minimum of two product containers to create a composite. Transfer about 1 gram of the sample composite into a porcelain crucible, and convert into a nearly colorless ash with the aid of sulfuric acid, heating, and ignition in a muffle furnace. Dissolve the residue in about 0.2 mL of nitric acid and a few mL of water, warming if necessary, and dilute with 100 mL of water. The solution imparts an intense yellow color to a non-luminous flame.
²Combine a minimum of two product containers to create a composite. Centrifuge 5 mL of the sample composite for 10 minutes, decant the supernatant layer, add 40 mL of methanol, and mix well. Filter the mixture using a glass-fiber paper (Whatman GF/A or equivalent) and a vacuum. Wash the residue successively, in the order named, with 10 mL of water, 10 mL of methanol, 10 mL of ethyl alcohol, and 10 mL of chloroform. The infrared spectrum of a potassium bromidedispersion of the dried residue (resin) (3.5 mg dried resin with 150 mg KBr) exhibits maxima only at the same wavelengths as that of a similar preparation of a previously approved lot of Amberlite IRP 69 Raw Material.

Each of Batch Nos. 1 and 2 of the above formulations were then split into A and B batches and tested for stability as packaged products in commercial scale lots of 500 mL bottles, 60 mL bottles, 120 mL enema bottles, and 200 mL enema bottles. The tables below represent the results of the tests as described in Table 7 for each of the aforementioned packaged products.

Packaged Product Test Results for Batch No. 1A, 60 mL Bottle

TABLE 8

| | Beginning of | End |
|---|---|---|
| Test | Packaging Run | of Packaging Run |
| Description | Conforms | Conforms |
| Identification A | — | Conforms |
| Identification B | — | Conforms |
| Potassium Exchange Capacity (mg/g) | 130 | 130 |
| Sodium Content (% w/v) | 2.71 | 2.61 |
| Methylparaben Assay (% LA) | 94.6 | 99.3 |
| Propylparaben Assay (% LA) | 92.2 | 97.5 |
| Solids Content (%) | 28.4 | 27.4 |
| Alcohol (%) | 0.09 | 0.09 |
| pH | 6.1 | 6.2 |
| Density (g/mL) | 1.119 | 1.117 |
| Resuspendability | Conforms | Conforms |
| Preservative Effectiveness | — | Pass |

—: not tested

Packaged Product Test Results for Batch No. 1B, 500 mL Bottle

TABLE 9

| Test | BOR | EOR |
|---|---|---|
| Description | Conforms | Conforms |
| Identification A | — | Conforms |
| Identification B | — | Conforms |
| Potassium Exchange Capacity (mg/g) | 131 | 131 |
| Sodium Content (% w/v) | 2.71 | 2.70 |
| Methylparaben Assay (% LA) | 102.7 | 101.1 |
| Propylparaben Assay (% LA) | 103.2 | 100.9 |
| Solids Content (%) | 28.4 | 28.3 |
| Alcohol (%) | 0.08 | 0.09 |
| pH | 6.2 | 6.1 |
| Density (g/mL) | 1.120 | 1.121 |
| Resuspendability | Conforms | Conforms |
| Preservative Effectiveness | — | Pass |

—: not tested

Packaged Product Test Results for Batch No. 2A, 120 mL Enema

TABLE 10

| Test | BOR | EOR |
|---|---|---|
| Description | Conforms | Conforms |
| Identification A | — | Conforms |
| Identification B | — | Conforms |
| Potassium Exchange Capacity (mg/g) | 126 | 122 |
| Sodium Content (% w/v) | 2.52 | 2.53 |
| Methylparaben Assay (% LA) | 97.8 | 101.1 |
| Propylparaben Assay (% LA) | 96.1 | 100.5 |
| Solids Content (%) | 26.5 | 26.4 |
| Alcohol (%) | 0.08 | 0.09 |
| pH | 6.0 | 6.1 |
| Density (g/mL) | 1.111 | 1.112 |
| Resuspendability | Conforms | Conforms |
| Preservative Effectiveness | — | Pass |

—: not tested

Packaged Product Test Results for Batch No. 2B, 200 mL Enema

TABLE 11

| Test | BOR | EOR |
|---|---|---|
| Description | Conforms | Conforms |
| Identification A | — | Conforms |
| Identification B | — | Conforms |
| Potassium Exchange Capacity (mg/g) | 126 | 124 |
| Sodium Content (% w/v) | 2.54 | 2.37 |
| Methylparaben Assay (% LA) | 96.6 | 97.8 |
| Propylparaben Assay (% LA) | 96.0 | 98.1 |
| Solids Content (%) | 26.8 | 25.9 |
| Alcohol (%) | 0.09 | 0.09 |
| pH | 6.1 | 6.1 |
| Density (g/mL) | 1.114 | 1.110 |
| Resuspendability | Conforms | Conforms |
| Preservative Effectiveness | — | Pass |

—: not tested

The above data demonstrates that the packaged products from commercial scale lots of the sorbitol free sodium polystyrene sulfonate suspension meet the intended specifications for all four configurations intended for supply to the marketplace.

Long Term Storage Stability

Samples of sodium polystyrene sulfonate suspension prepared according to the above example (15.0 g/60.0 mL) were tested for a number of stability parameters including, pH, potassium exchange capacity, sodium content, methylparaben content, propylparaben content, and resuspendability when stored under conditions of 25° C. and 60% relative humidity (RH) for periods of 3, 6, 9, 12 and 18 months. Samples were stored in 60 mL and 500 mL containers and also 120 mL and 200 mL enema containers, stored on their sides.

TABLE 12
60 mL Containers: Stored on Side (25° C./60% RH)
Sodium Polystyrene Sulfonate Suspension 15 g/60.0 mL

| | Acceptance | Timepoint | | | | | |
|---|---|---|---|---|---|---|---|
| Test | Criteria | Initial | 3 mo | 6 mo | 9 mo | 12 mo | 18 mo |
| Description | Conforms | Conforms | Conforms | Conforms | Conforms | Conforms | Conforms |
| pH | 4.0–7.5 | 6.2 | 7.3 | 7.0 | 6.9 | 7.1 | 7.2 |
| Potassium | 110 to 135 | 130 | 133 | 134 | 131 | 126 | 129 |
| Exchange | | | | | | | |
| Capacity | | | | | | | |
| (mg/g) | | | | | | | |
| Sodium Content | 2.35%–2.88% | 2.61 | 2.78 | 2.82 | 2.73 | 2.70 | 2.76 |
| (% w/v) | | | | | | | |
| Methylparaben | 60.0–120.0% | 99.3 | 99.7 | 99.9 | 103.1 | 97.5 | 98.7 |
| (% LA) | | | | | | | |
| Propylparaben | 60.0–120.0% | 97.5 | 101.3 | 97.2 | 105.6 | 98.1 | 99.3 |
| (% LA) | | | | | | | |
| Resuspendability | Resuspends | Resuspends | Resuspends | Resuspends | Resuspends | Resuspends | Resuspends |

TABLE 13
500 mL Containers: Stored on Side (25° C./60% RH)
Sodium Polystyrene Sulfonate Suspension 15.0 g/60.0 mL

| | Acceptance | Timepoint | | | | | |
|---|---|---|---|---|---|---|---|
| Test | Criteria | Initial | 3 mo | 6 mo | 9 mo | 12 mo | 18 mo |
| Description | Conforms | Conforms | Conforms | Conforms | Conforms | Conforms | Conforms |
| pH | 4.0–7.5 | 6.1 | 7.0 | 6.9 | 7.0 | 7.0 | 7.2 |
| Potassium | 110 to 135 | 131 | 132 | 132 | 130 | 130 | 131 |
| Exchange | | | | | | | |
| Capacity | | | | | | | |
| (mg/g) | | | | | | | |
| Sodium Content | 2.35%–2.88% | 2.70 | 2.91 | 2.80 | 2.72 | 2.83 | 2.76 |
| (% w/v) | | | 2.86 | | | | |
| | | | 2.84 | | | | |
| Methylparaben | 60.0–120.0% | 101.1 | 101.3 | 102.3 | 99.9 | 101.1 | 97.2 |
| (% LA) | | | | | | | |
| Propylparaben | 60.0–120.0% | 100.9 | 102.3 | 99.8 | 102.6 | 102.3 | 96.0 |
| (% LA) | | | | | | | |
| Resuspendability | Resuspends | Resuspends | Resuspends | Resuspends | Resuspends | Resuspends | Resuspends |

TABLE 14
120 mL Enema Containers: Stored on side (25° C./60% RH)
Sodium Polystyrene Sulfonate Suspension 15.0 g/60.0 mL

| | Acceptance | Timepoint | | | | | |
|---|---|---|---|---|---|---|---|
| Test | Criteria | Initial | 3 mo | 6 mo | 9 mo | 12 mo | 18 mo |
| Description | Conforms | Conforms | Conforms | Conforms | Conforms | Conforms | Conforms |
| pH | 4.0–7.5 | 6.1 | 7.0 | 7.1 | 7.0 | 7.1 | 7.3 |
| Potassium | 110 to 135 | 122 | 130 | 127 | 126 | 126 | 126 |
| Exchange | | | | | | | |
| Capacity | | | | | | | |
| (mg/g) | | | | | | | |
| Sodium Content | 2.35%–2.88% | 2.53 | 2.68 | 2.61 | 2.64 | 2.65 | 2.65 |
| (% w/v) | | | | | | | |
| Methylparaben | 60.0–120.0% | 101.1 | 101.8 | 101.0 | 101.6 | 99.2 | 99.6 |
| (% LA) | | | | | | | |
| Propylparaben | 60.0–120.0% | 100.5 | 102.2 | 98.8 | 103.1 | 99.4 | 100.0 |
| (% LA) | | | | | | | |
| Resuspendability | Resuspends | Resuspends | Resuspends | Resuspends | Resuspends | Resuspends | Resuspends |

TABLE 15
200 mL Enema Containers (25° C./60% RH)
Sodium Polystyrene Sulfonate Suspension 15.0 g/60.0 mL

| | Acceptance | Timepoint | | | | | |
|---|---|---|---|---|---|---|---|
| Test | Criteria | Initial | 3 mo | 6 mo | 9 mo | 12 mo | 18 mo |
| Description | Conforms | Conforms | Conforms | Conforms | Conforms | Conforms | Conforms |
| pH | 4.0–7.5 | 6.1 | 6.9 | 7.2 | 7.0 | 7.0 | 7.3 |
| Potassium | 110 to 135 | 124 | 128 | 123 | 119 | 126 | 126 |
| Exchange | | | | | | | |
| Capacity | | | | | | | |
| (mg/g) | | | | | | | |
| Sodium Content | 2.35%–2.88% | 2.37 | 2.65 | 2.48 | 2.48 | 2.68 | 2.65 |
| (% w/v) | | | | | | | |
| Methylparaben | 60.0–120.0% | 97.8 | 101.2 | 97.5 | 97.1 | 100.5 | 99.2 |
| (% LA) | | | | | | | |
| Propylparaben | 60.0–120.0% | 98.1 | 102.5 | 94.7 | 98.6 | 101.2 | 98.8 |
| (% LA) | | | | | | | |
| Resuspendability | Resuspends | Resuspends | Resuspends | Resuspends | Resuspends | Resuspends | Resuspends |

As shown in the above tables, each of the samples of sodium polystyrene sulfonate suspension prepared in accordance with the present invention meets the acceptance criteria for each of the parameters including, pH, potassium exchange capacity, sodium content, methylparaben content, propylparaben content, and resuspendability when stored under conditions of 25° C. and 60% relative humidity (RH) from the time of formulation and for periods of 3, 6, 9, 12 and 18 months.

Having described the invention in detail and by reference to preferred embodiments thereof, it will be apparent that modifications and variations are possible without departing from the scope of the invention defined hereinbefore.

What is claimed:

1. A pharmaceutically acceptable formulation of sodium polystyrene sulfonate for treatment of hyperkalemia comprising a pharmaceutically acceptable amount of sodium polystyrene sulfonate a suspending agent and a vehicle wherein the formulation is free of sorbitol.

2. The formulation of claim 1 wherein the vehicle is selected from water, propylene glycol, glycerin, polyethylene glycol, polyglyceryl-6 oleate and mixtures thereof.

3. The formulation of claim 2 wherein the vehicle is water.

4. The formulation of claim 1 wherein the suspending agent is selected from gums, cellulosics, clays, carbomer NF, gelatin, polyethylene glycols and lecithin.

5. The formulation of claim 4 wherein the suspending agent is magnesium aluminum silicate clay.

6. The formulation of claim 1 which is a ready-to-use formulation.

7. The formulation of claim 1 wherein the formulation has the following composition:

| Sodium Polystyrene Sulfonate | 15.0 g/60.0 mL | |
|---|---|---|
| Solvent/vehicle | 0–99% | w/v |
| Preservative | 0–5% | w/v |
| Suspending Agent | 0–50% | w/v |
| Sweetening Agent | 0–5% | w/v |
| Buffers/pH Adjusters | 0–2% | w/v |
| Flavors | 0–3% | v/v |

8. The formulation of claim 7 having the following composition:

| | | |
|---|---|---|
| Sodium Polystyrene Sulfonate | 15.0 | g |
| Propylene Glycol, USP | 10% | w/v |
| Methylparaben, NF | 0.18% | w/v |
| Propylparaben, NF | 0.02% | w/v |
| Magnesium Aluminum Silicate, NF | 1.75% | w/v |
| Saccharin Sodium, USP | 0.025% | w/v |
| Citric Acid, USP (Anhydrous, Fine Gran.) | 0.125% | w/v |
| Flavor | 0.75% | v/v |
| | |
|---|---|
| Water, Purified USP | QS |

9. A storage stable pharmaceutically acceptable formulation of sodium polystyrene sulfonate for treatment of hyperkalemia comprising a pharmaceutically acceptable amount of sodium polystyrene sulfonate and a suspending agent wherein the formulation is free of sorbitol.

10. The formulation of claim 9 wherein the composition is stable when stored under temperatures of about 25° C. and a relative humidity of about 60% to for a period of up to at least 18 months.

11. The formulation of claim 9 wherein the composition is stable when stored under temperatures of about 40° C. and a relative humidity of about 75% to for a period of up to at least 3 months.

12. The formulation of claim 9 wherein the formulation meets FDA stability requirements for sodium polystyrene sulfonate suspensions for one of more of pH, potassium exchange capacity, sodium content, methylparaben content, propylparaben content, and resuspendability.

13. A method of treating a patient suffering from hyperkalemia by administering a formulation according to claim 1 to a patient in need thereof.

14. The method of claim 13 wherein the formulation is administered orally.

15. The method of claim 13 wherein the formulation is administered rectally.

16. A method of treating a patient suffering from hyperkalemia by administering a formulation according to claim 8 to a patient in need thereof.

17. The method of claim 16 wherein the formulation is administered orally.

18. The method of claim 16 wherein the formulation is administered rectally.

19. A process for preparing a formulation comprising sodium polystyrene sulfonate and a suspending agent wherein the formulation is free of sorbitol, wherein the process comprises dispersion of the sodium polystyrene sulfonate and the suspending agent through a rotor-stator system.

20. The process of claim 19 wherein the suspending agent is magnesium aluminum silicate.
